(19) United States
(12) Patent Application Publication
(10) Pub. No.: US 2005/0101896 A1
(43) Pub. Date: May 12, 2005

(54) Cervical collar having enhanced lateral support

(76) Inventor: Salvatore Calabrese, Philadelphia, PA (US)

Correspondence Address: SCHNADER HARRISON SEGAL & LEWIS, LLP, 1600 MARKET STREET, SUITE 3600, PHILADELPHIA, PA, 19103, US

(21) Appl. No.: 10/507,058

(22) PCT Filed: Mar. 11, 2003
(86) PCT No.: PCT/US03/07655

Related U.S. Application Data
(60) Provisional application No. 60/363,416, filed on Mar. 11, 2002.

(51) Int. Cl. A61F 5/00

(52) U.S. Cl. 602/18

(57) ABSTRACT

A cervical collar (10) having a front collar portion (12) having a chin support portion (40) and generally conforming to the neck, shoulders, and mandible of a wearer. The front collar portion (12), is fabricated from a soft flexible material and a lateral reinforcing support member (30) disposed in the neck region below the mandible bone of the wearer. The support member (30) is fabricated from a substantially incompressible material.

### FIELD OF THE INVENTION

This invention relates generally to cervical collars, and more particularly to cervical collars having enhanced lateral support.

### BACKGROUND OF THE INVENTION

Head immobilization is necessary when a person has suffered trauma to his head, neck, and/or spine. Various cervical collars are generally known to provide such head immobilization. One such collar is a two-piece cervical collar fabricated from a soft, flexible, light-weight material, for example a non-injection molded plastic material such as polyethylene or polyurethane. These cervical collars provide the necessary head immobilization needed for most neck and head injuries and also provide a increased degree of comfort due to their soft flexible construction. However, in some instances, enhanced lateral support may be necessary to provide an increased degree of head immobilization in the lateral direction and/or rotational support.

### SUMMARY

A cervical collar comprising a front collar portion having a chin support portion and generally conforming to the neck, shoulders, and mandible of a wearer. The front collar portion is fabricated from a soft flexible material and a lateral reinforcing support member disposed in the neck region below the mandible bone of the wearer. The support member is fabricated from a substantially incompressible material.

The support member can be substantially flat and can have at least one rounded corner which may engage the mandible of the wearer for limiting the rotating of the wearer's neck. Typically, the support plates are positioned parallel to the wearer's jawbone. The support member can be attached to the front collar portion with one or more rivets. A plurality of the support members may be provided on each lateral side of the wearer's neck.

In one form of the invention, the support member is fabricated from a lightweight and rigid material for providing sufficient lateral support to the member's head. The support plate may also be fabricated from a substantially incompressible material.

In another form of the invention, the front collar portion is fabricated from a foam material. A fabric fastener for connecting the front collar half to a rear collar half may be employed. The fabric may be attached to the front collar portion with a rivet.

In one form of the invention, a plurality of support plates may be provided to increase the lateral support. In addition, the support member has a color which is indicative of a material property of the support member.

### BRIEF DESCRIPTION OF THE DRAWINGS

For the purpose of illustrating the invention, there is shown in the drawings embodiments which are presently preferred; it being understood, however, that this invention is not limited to the precise arrangement and instrumentalities shown.

FIG. 1 is a front elevational view of a preferred embodiment of a cervical collar of the present invention

FIG. 2 is a side view of the cervical collar of FIG. 1;

FIG. 3 is a back elevational view of the cervical collar of FIG. 1 with a portion of the fabric fastener folded back;

FIG. 4 is a side view of the cervical collar of FIG. 1 taken along line V-V;

### DETAILED DESCRIPTION OF THE PREFERRED EMBODIMENT

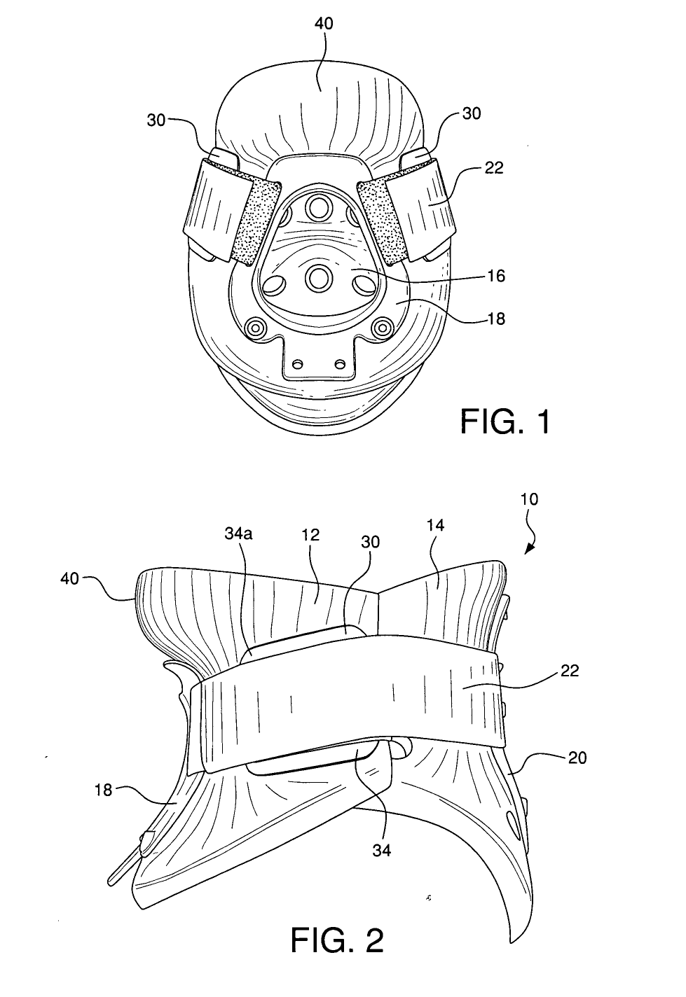

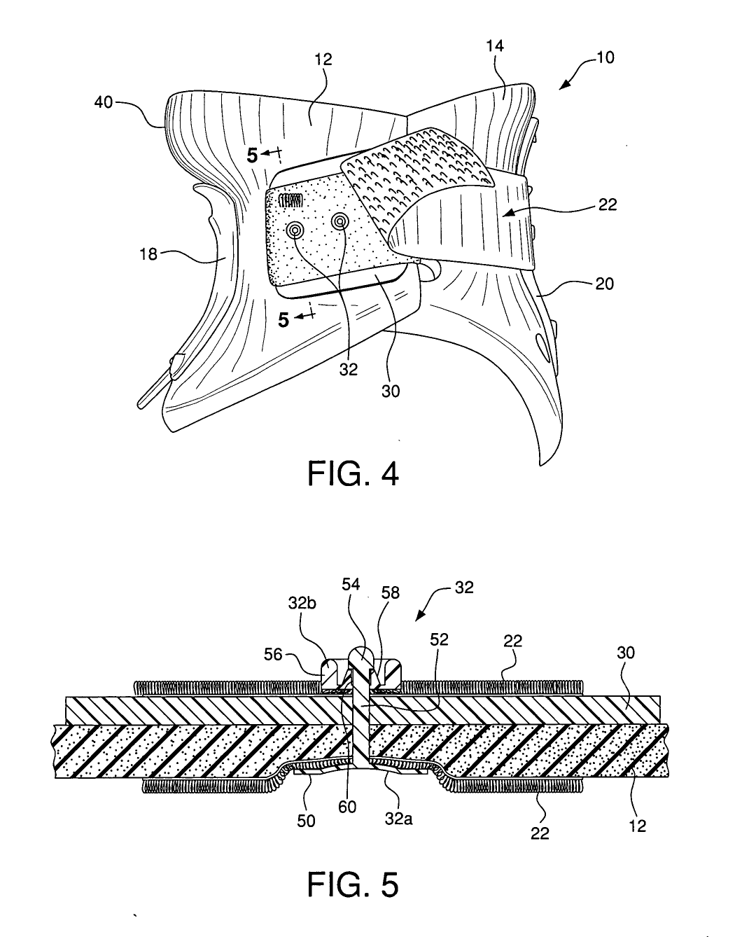

Referring to the drawings, wherein like numerals indicate like elements, there is shown in FIGS. 1-5 a cervical collar 10 having a front half collar portion 12 and a separate rear half portion 14. The cervical collar of the present invention is described generally in U.S. Pat. Nos. 3,756,226 and 4,677,962 to Calabrese which are hereby incorporated by reference herein.

The front half collar portion 12 is for application to the front of a wearer's neck region and generally comprises a U-shaped body preferably fabricated from a soft, flexible, light-weight material which is preferably non-toxic, hypo-allergenic, latex-free, water-resist and buoyant in water, for example a non-injection molded closed cell polymeric plastic material such as polyethylene or polyurethane. The front collar portion 12 has a chin support portion 40 for supporting the wearer's chin and is generally shaped to conform to the front of the wearer's chin, mandible, neck, shoulders, and upper chest.

Similarly, the rear half collar portion 14 is for application to the rear of a wearer's neck region and is fabricated from a similar material as that of front half portion 12. The rear half collar portion is generally shaped to conform to the rear of the wearer's chin, mandible, neck, shoulders, and back/spine.

The front collar half 12 can be provided with an opening 16 below the chin support 40 for providing access to the tracheal region of the person's neck and for otherwise providing ventilation to the neck region.

Referring back to FIG. 1, the front half collar portion 12 is also provided with a rigid reinforcing support member 18 fabricated from an incompressible plastic material which is stiff and generally inflexible. Typically, the support member 18 can also have a tracheal opening similar to opening 16.

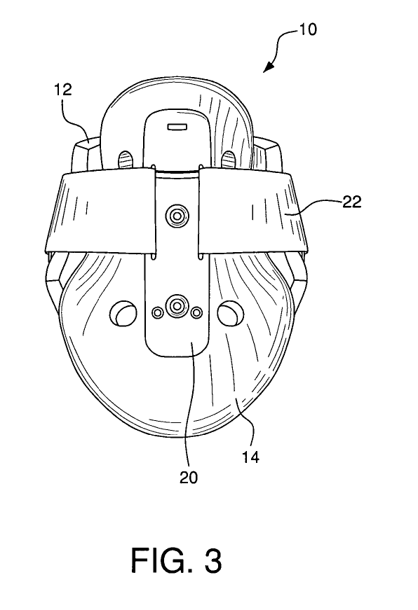

Similarly, as best shown in FIG. 3, the rear half portion 14 is provided with a similar reinforcing support member 20 which is fabricated from the same material as the support member 18. The front and rear support members 18 and 20 function to limit or decrease the flexion and extension of a wearer's head in the front and rear direction respectively. The support members 18 and 20 can also be provided with additional connection elements, such as openings and/or slots, as is well known in the art. In addition, the support members 18 and 20 can also be provided with slots, of the like, for attachment of fabric fasteners for joining the front and rear collar halves around the neck of a wearer.

In addition, a hook and loop fabric fastener 22 is provided for connecting the front and rear halves of the collar together. One strip of fabric fastener strip is connected to the rear collar half, typically to the rear support member 20. Similarly, a second fabric fastener strips are connected to the front collar half 12, typically to the front support member 18. The free ends of the fastener strips are attached to secure the collar halves together.

The cervical collar 10 is provided with lateral reinforcement support members or plates 30 for reducing the range of lateral and rotational motion of a wearer's head. The lateral reinforcement support members 30 are located on both sides of the cervical collar 10 in the wearer's neck region and they may be attached to the collar 10 by any suitable means, such as by rivets 32, adhesive, sonic welding, molding in place, or the like. In addition, the lateral reinforcing members 30 can be fabricated by any lightweight and rigid material to achieve the desired lateral and rotational support.

Moreover, the fabric fastener strips can be fastened to the collar halves in the same manner as the lateral supports 30. In the embodiment shown, rivets 32 fasten the fasten strips and the support members 30 to the front collar half portion 12.

As best seen in FIG. 5, one preferred rivet design is illustrated. The rivet 32 comprise a male portion 32a and a female portion 32b. The male portion includes an enlarged end portion and an upstanding male member 52 having an enlarged rounded head 54. The female portion 32b has an upstanding peripheral wall 56, a medial upstanding circular wall 58, and a central opening 60 inside the medial wall 58. To engage the male and female rivet portions, the enlarged head 54 of the male member 52 is passed through the medial opening 60 of the female portion and the bottom radial surface enlarged head is trapped on the medial wall 58 to prevent removal.

Although the supports 30 are shown as flat plates with rounded corners 34, it is contemplated that the lateral reinforcement support members 30 can be of various thicknesses, and rigidities to provide the desired degree of lateral support. For example, the lateral reinforcement support members 30 can be fabricated as a thin-walled structure having additional structural reinforcing supports or ribs (not shown). It is also contemplated that the lateral reinforcing support members 30 can be fabricated from any lightweight and rigid material that can achieve the desired support properties described herein, such as polypropylene or the like.

It is also contemplated that the lateral reinforcement members 30 can be offered in various sizes and rigidities so that the fitter, user, or physical therapist can vary the supports 30 by changing the number of supports used or the rigidity and/or size of the support(s) used to vary the amount of lateral and rotational support provided thereby.

It should be understood that the lateral reinforcement members 30 primarily serve to decrease the lateral flexion and rotation of the wearer's head over and above the support already provided by the foam collar 10. This lateral support can be used to limit the range of lateral and rotational motion or to provide resistance against lateral and rotational motion of the head so as to provide the necessary resistance to strengthen the neck muscles of the wearer employing the cervical collar 10 in a physical therapy or exercise regime.

The lateral reinforcing members 30 are located on the cervical collar 10 in the neck region of the wearer and below the mandible bone of the wearer such that the upper front corner 34a engages the mandible of the wearer to limit the rotation of the wearer's head. As such, it is contemplated that the lateral supports 30 are positioned parallel to the wearer's jawbone.

It is also contemplated that the lateral reinforcement support members 30 can be fabricated together with the front support 18 to facilitate fabrication, manufacture, and assembly of the collar. Or, alternatively, the lateral support members 30 can be fabricated separately to facilitate removal or replacement thereof as described above.

It is also contemplated that the lateral reinforcement supports 30 can be manufactured in a wide range of colors or can be manufactured using different color schemes to compliment or enhance the color of the cervical collar body itself.

The present invention may be embodied in other specific forms about departing from the spirit or essential attributes thereof and, accordingly, reference should be made to the appended claims, rather than to the forgoing specification, as indicating the scope of the invention.

1. A cervical collar comprising:
a front collar portion having a chin support portion and generally conforming to the neck, shoulders, and mandible of a wearer, the front collar portion being fabricated from a soft flexible material; and
a lateral reinforcing support member disposed in the neck region below the mandible bone of the wearer, the support member being fabricated from a substantially incompressible material.

2. The cervical collar of claim 1, wherein the support member has a generally rectangular shape forming a plate.

3. The cervical collar of claim 2, wherein the plate is substantially flat.

4. The cervical collar of claim 3, wherein the support plate has at least one rounded corner.

5. The cervical collar of claim 4, wherein the at least one rounded corner engages the mandible of the wearer for limiting the rotating of the wearer's neck.

6. The cervical collar of claim 5, wherein the support plate is positioned parallel to the wearer's jawbone.

7. The cervical collar of claim 1, wherein the support member is fabricated from a lightweight and rigid material for providing sufficient lateral support to the wearer's head.

8. The cervical collar of claim 1, wherein the front collar portion is fabricated from a foam material.

9. The cervical collar of claim 1, wherein the support member is attached to the front collar portion with at least one rivet.

10. The cervical collar of claim 9 further comprising a fabric fastener for connecting the front collar half to a rear collar half, and wherein the fabric fastener is attached to the front collar portion with the rivet.

11. The cervical collar of claim 1, wherein the support member is a plate having at least one rounded corner.

12. The cervical collar of claim 1, wherein the support plate is flat and rectangular.

13. The cervical collar of claim 12, wherein the support plate is fabricated from a substantially incompressible material.

14. The cervical collar of claim 11, wherein a plurality of the support plates are provided to increase the lateral support.

15. The cervical collar of claim 1, wherein the support member is a plate having at least one rounded corner.

16. The cervical collar of claim 1, wherein the support member has a color which is indicative of a material property of the support member.

17. The cervical collar of claim 1, wherein a plurality of the support members are provided, at least one support member being provided for each lateral side of the wearer's neck.

18. A method of exercising neck muscles comprising:
providing a cervical collar comprising a front collar portion having a chin support portion and generally conforming to the neck, shoulders, and mandible of a wearer, the front collar portion being fabricated from a soft flexible material; and a lateral reinforcing support member disposed in the neck region below the mandible bone of the wearer, the support member being fabricated from a substantially incompressible material;
applying the cervical collar to a wearer's neck; and
moving the wearer's head to one side to strengthen the wearer's neck muscles against the resistance provided by the lateral support member.

19. A method of fitting a cervical collar comprising the steps of:
providing a cervical collar comprising a front collar portion having a chin support portion and generally conforming to the neck, shoulders, and mandible of a wearer, the front collar portion being fabricated from a soft flexible material;
fitting a lateral reinforcing support member to the front collar portion in the neck region below the mandible bone of the wearer, the support member being fabricated from a substantially incompressible material;
testing the lateral support provided by the lateral support member;
fitting at least one additional lateral support members to the front portion depending upon the results of the test.
